(19) United States
(12) Patent Application Publication
(10) Pub. No.: US 2023/0363403 A1
(43) Pub. Date: Nov. 16, 2023

(54) LACTIC ACID BACTERIA STARTER, METHOD FOR PRODUCING FERMENTED MILK, AND FERMENTED MILK

(71) Applicant: MEIJI CO., LTD., Tokyo (JP)

(72) Inventors: Hanae TSUCHIHASHI, Hachioji-shi, Tokyo (JP); Eri YAMAMOTO, Hachioji-shi, Tokyo (JP); Atsuko UENO, Hachioji-shi, Tokyo (JP); Yoshitaka KAWAI, Hachioji-shi, Tokyo (JP); Ayumi ICHIKAWA, Hachioji-shi, Tokyo (JP)

(73) Assignee: MEIJI CO., LTD., Tokyo (JP)

(21) Appl. No.: 18/021,897

(22) PCT Filed: Aug. 20, 2021
(86) PCT No.: PCT/JP2021/030515

§ 371 Date: Feb. 17, 2023

(30) Foreign Application Priority Data
Aug. 21, 2020 (JP) 2020-139992

(51) Int. Cl. A23C 9/123

(52) U.S. Cl. CPC A23C 9/1238; A23C 9/1234; A23V 2400/145; A23V 2400/147; A23V 2400/165; A23V 2400/169; A23V 2400/175; A23V 2400/113; A23V 2400/115; A23V 2400/151; A23V 2400/179; A23V 2400/181; A23V 2400/167; A23V 2400/143; A23V 2400/173; A23V 2400/121; A23V 2400/157

(57) ABSTRACT

The present invention provides a lactic acid bacteria starter comprising: Streptococcus thermophilus; and a lactic acid bacterium of the Lactobacillaceae other than Lactobacillus delbrueckii.

### TECHNICAL FIELD

The present invention relates to a lactic acid bacteria starter, a method for producing fermented milk, and fermented milk, and more particularly to a lactic acid bacteria starter, a method for producing fermented milk using the same, and fermented milk obtained thereby.

### BACKGROUND ART

For example, in Japan's “Ministerial Ordinance on Milk and Milk Products Concerning Compositional Standards, etc. (Ministerial Ordinance on Milk and Milk Products)”, fermented milk is defined as “products which are obtained by fermenting milk, or milk, etc. containing an equal or greater amount of milk solids-not-fat with lactic acid bacteria or yeast and then forming a paste or liquid, or the frozen product”, and the lactic acid bacteria or yeast is called a starter for the production of fermented milk. Representative examples of such fermented milk include yogurt such as set type yogurt (solid fermented milk), soft type yogurt (pasty fermented milk), and drink type yogurt (liquid fermented milk).

In the “Codex Alimentarius (FAO (Food and Agriculture Organization of the United Nations)/WHO (World Health Organization))”, which is a food standard shared by the international community, yogurt is defined as “any food that is made through the process of lactic acid fermentation combining Lactobacillus delbrueckii subsp. bulgaricus (Bulgarian bacterium) and Streptococcus thermophilus (thermophilus bacterium)”. Such yogurt is characterized by having a refreshing sourness and fermented aroma. Conventionally, yogurts using these Bulgarian bacterium and thermophilus bacterium have commonly been used.

For example, International Publication No. WO2018/151249 (PTL 1) describes a method for producing fermented milk comprising a step of adding a lactic acid bacteria starter to a raw material mix to obtain a fermented milk base and a fermentation step of fermenting the fermented milk base at 35 to 50° C., and states that the lactic acid bacteria starter includes the Bulgarian bacterium and thermophilus bacterium. Also, for example, International Application Japanese-Phase Publication No. 2015-518374 (PTL 2) describes the use of certain Streptococcus thermophilus strains and Lactobacillus delbrueckii subsp. bulgaricus strains for the production of fermented milk products.

On the other hand, the Codex Alimentarius defines fermented milk using lactic acid bacteria other than the Bulgarian bacterium as “alternate culture yogurt”. In addition, fermented milk can be produced by using only one type of lactic acid bacterium, and traditional fermented milks using yeasts also exist in various parts of the world. These fermented milks are known to have unique flavors that are differentiated from conventional yogurt according to the Codex Alimentarius, and are attracting attention because there is, for example, an increasing demand for mild flavor such as with low sourness but with milkiness and sweetness as consumer tastes and eating methods of yogurt have diversified in recent years. As the alternate culture yogurt, for example, Meiji Co., Ltd. manufactures and sells “Meiji Hokkaido Tokachi Rich & Mild (taste) yogurt” using Lactobacillus delbrueckii, which is of the same species as the Bulgarian bacterium but is of a different subspecies.

### CITATION LIST

### Patent Literature

[PTL 1] International Publication No. WO2018/151249

[PTL 2] International Application Japanese-Phase Publication No. 2015-518374

### SUMMARY OF INVENTION

### Technical Problem

However, the present inventors conducted research on fermented milk and a lactic acid bacteria starter for producing the same, and found that fermented milk using one type of lactic acid bacterium alone had a flavor different from that using Lactobacillus delbrueckii (L. delbrueckii) such as the Bulgarian bacterium due to the bacterial species-specific metabolite pattern, but the flavor was not always favorable in many cases. The present inventors also found that when one type of lactic acid bacterium was used alone, it took longer to complete fermentation than the case of using the Bulgarian bacterium and thermophilus bacterium. For example, when a lactic acid bacterium belonging to the Lactobacillaceae (the family Lactobacillaceae) other than Lactobacillus delbrueckii was used alone for fermentation, it took time to complete the fermentation (for example, the pH became 4.5 or less), and at the shortest the fermentation time was more than 11 hours, and most of the lactic acid bacteria did not complete the fermentation within 24 hours. In the production of fermented milk, if it takes too long to complete fermentation, in addition to the problem of deterioration in production efficiency, there is a tendency for problems such as an increase in the risk of contamination to hinder stable production of fermented milk and a deterioration in flavor.

The present invention has been made in view of the above-mentioned problems of the prior art, and aims to provide a novel lactic acid bacteria starter that can stably produce fermented milk that completes fermentation in a sufficiently short time and has a flavor characteristic differentiated from conventional fermented milk (preferably yogurt), “mild flavor with low sourness but with milkiness and sweetness”, a method for producing fermented milk using the same, and fermented milk obtained thereby.

### Solution to Problem

As a result of intensive research to achieve the above object, the present inventors have found that by combining lactic acid bacteria of the Lactobacillaceae other than Lactobacillus delbrueckii with Streptococcus thermophilus (thermophilus bacterium), the time until completion of fermentation (preferably, the time until pH becomes 4.5 or less) can be sufficiently shortened. Furthermore, the present inventors have found that the fermented milk obtained by this combination particularly has a favorable flavor differentiated from conventional fermented milk, “mild flavor with low sourness but with milkiness and sweetness”. Thus, the present invention has been completed.

The aspects of the present invention obtained from such findings are as follows.

[1]

A lactic acid bacteria starter comprising: Streptococcus thermophilus; and a lactic acid bacterium of the Lactobacillaceae other than Lactobacillus delbrueckii.

[2]

The lactic acid bacteria starter according to [1], wherein the Streptococcus thermophilus is a strain carrying a prtS gene.

[3]

The lactic acid bacteria starter according to [1] or [2] , wherein the lactic acid bacterium of the Lactobacillaceae is least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

[4]

The lactic acid bacteria starter according to any one of [1] to [3], wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.

[5]

A method for producing fermented milk, comprising: a fermentation step of adding the lactic acid bacteria starter according to any one of [1] to [4] to a milk preparation solution containing raw material milk and fermenting the mixture to obtain fermented milk.

[6]

A method for producing fermented milk, comprising: a fermentation step of adding Streptococcus thermophilus and a lactic acid bacterium of the Lactobacillaceae other than Lactobacillus delbrueckii to a milk preparation solution containing raw material milk and fermenting the mixture to obtain fermented milk.

[7]

The method for producing fermented milk according to [6], wherein the Streptococcus thermophilus is a strain carrying a prtS gene.

The method for producing fermented milk according to [6] or [7], wherein the lactic acid bacterium of the Lactobacillaceae is at least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

[9]

The method for producing fermented milk according to any one of [6] to [8], wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.

[10]

A fermented milk comprising: Streptococcus thermophilus; and a lactic acid bacterium of the Lactobacillaceae other than Lactobacillus delbrueckii.

[11]

The fermented milk according to [10], wherein the Streptococcus thermophilus is a strain carrying a prtS gene.

[12]

The fermented milk according to [10] or [11], wherein the lactic acid bacterium of the Lactobacillaceae is at least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

[13]

The fermented milk according to any one of [10] to [12], wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.

[14]

A method for promoting fermentation of fermented milk, comprising: a fermentation step of adding the lactic acid bacteria starter according to any one of [1] to [4] to a milk preparation solution containing raw material milk and fermenting the mixture to obtain fermented milk.

[15]

A method for promoting fermentation of fermented milk, comprising: a fermentation step of adding Streptococcus thermophilus and a lactic acid bacterium of the Lactobacillaceae other than Lactobacillus delbrueckii to a milk preparation solution containing raw material milk and fermenting the mixture to obtain fermented milk.

[16]

A method for improving flavor of fermented milk, comprising: a fermentation step of adding the lactic acid bacteria starter according to any one of [1] to [4] to a milk preparation solution containing raw material milk and fermenting the mixture to obtain fermented milk.

[17]

A method for improving flavor of fermented milk, comprising: a fermentation step of adding Streptococcus thermophilus and a lactic acid bacterium of the Lactobacillaceae other than Lactobacillus delbrueckii to a milk preparation solution containing raw material milk and fermenting the mixture to obtain fermented milk.

[18]

The method according to [15] or [17], wherein the Streptococcus thermophilus is a strain carrying a prtS gene.

[19]

The method according to [15], [17], or [18], wherein the lactic acid bacterium of the Lactobacillaceae is at least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

[20]

The method according to any one of [15], [17], [18], and [19], wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.

### Advantageous Effects of Invention

The present invention makes it possible to provide a novel lactic acid bacteria starter that can stably produce fermented milk that completes fermentation in sufficiently short time and has a flavor characteristic differentiated from conventional fermented milk, “mild flavor with low sourness but with milkiness and sweetness”, a method for producing fermented milk using the same, and fermented milk obtained thereby.

### BRIEF DESCRIPTION OF DRAWINGS

FIG. 1 is a graph showing fermentation time when using S. thermophilus prtS(−) and S. thermophilus prtS(+) for the lactic acid bacteria of the Lactobacillaceae other than L. delbrueckii listed in Table 1.

FIG. 2 is a scatter diagram showing the relationship between the first principal component score (PC1) and the second principal component score (PC2) in result “(1) All Water-Soluble Components and Sensory Evaluation”, encircling the case of using S. thermophilus and the case of fermentation using lactic acid bacteria of the Lactobacillaceae alone without using S. thermophilus.

FIG. 3 is a scatter diagram encircling the case of using L. delbrueckii and the case of using the others for lactic acid bacteria of the Lactobacillaceae in FIG. 2.

FIG. 4 is a scatter diagram for the all water-soluble components and sensory evaluation items corresponding to plot positions of the samples of FIGS. 2 and 3 in result “(1) All Water-Soluble Components and Sensory Evaluation”.

FIG. 5 is a scatter diagram showing the relationship between the first principal component score (PC1) and the second principal component score (PC2) for the all water-soluble components and sensory evaluation values when using the lactic acid bacteria of the Lactobacillaceae other than L. delbrueckii in result “(1) All Water-Soluble Components and Sensory Evaluation”, encircling the case of using S. thermophilus prtS(−) and S. thermophilus prtS(+).

FIG. 6 is a scatter diagram for the all water-soluble components and sensory evaluation items corresponding to plot positions of the samples in FIG. 5 in result “(1) All Water-Soluble Components and Sensory Evaluation”.

FIG. 7 is a scatter diagram showing the relationship between the first principal component score (PC1) and the second principal component score (PC2) in result “(2) Aroma Compounds and Sensory Evaluation”, encircling the case of using S. thermophilus and the case of fermentation using lactic acid bacteria of the Lactobacillaceae alone without using S. thermophilus.

FIG. 8 is a scatter diagram encircling the case of using L. delbrueckii and the case of using the others for lactic acid bacteria of the Lactobacillaceae in FIG. 7.

FIG. 9 is a scatter diagram for the items of aroma compounds and sensory evaluation corresponding to plot positions of the samples of FIGS. 7 and 8 in result “(2) Aroma Compounds and Sensory Evaluation”.

FIG. 10 is a scatter diagram showing the relationship between the first principal component score (PC1) and the second principal component score (PC2) for the aroma compounds and sensory evaluation values when using the lactic acid bacteria of the Lactobacillaceae other than L. delbrueckii in result “(2) Aroma Compounds and Sensory Evaluation”, encircling the case of using S. thermophilus prtS(−) and S. thermophilus prtS(+).

FIG. 11 is a scatter diagram for the items of aroma compounds and sensory evaluation corresponding to plot positions of the samples in FIG. 10 in result “(2) Aroma Compounds and Sensory Evaluation”.

FIG. 12 is a graph showing fermentation time in the case of using S. thermophilus prtS (−), in the case of using S. thermophilus prtS(+), and in the case of not using S. thermophilus for the lactic acid bacteria of the Lactobacillaceae listed in Table 5.

### DESCRIPTION OF EMBODIMENTS

The present invention will now be described in detail with reference to preferred embodiments thereof.

### Lactic Acid Bacteria Starter

A lactic acid bacteria starter of the present invention comprises: Streptococcus thermophilus and a lactic acid bacterium of the Lactobacillaceae other than Lactobacillus delbrueckii. The lactic acid bacteria starter of the present invention can be suitably used for the method for producing fermented milk, the method for promoting fermentation of fermented milk, and the method for improving flavor of fermented milk, according to the present invention described below.

### Streptococcus thermophilus

The Streptococcus thermophilus according to the present invention (in the present specification, sometimes referred to as “S. thermophilus” or “thermophilus bacterium”) is not particularly limited, and may be used singly or in combination of two or more types. In the present invention, the use of S. thermophilus makes it possible to obtain fermented milk that can significantly shorten the time until fermentation completion and has a flavor characteristic differentiated from conventional fermented milk (for example, mild flavor with low sourness but with milkiness and sweetness), compared with the case of, for example, single use of the following lactic acid bacteria of the Lactobacillaceae other than Lactobacillus delbrueckii.

The S. thermophilus according to the present invention preferably has the prtS gene. By using S. thermophilus having the prtS gene (hereinafter sometimes referred to as “S. thermophilus prtS(+)”), the time until fermentation completion can be even shortened, and the flavor of fermented milk tends to be further improved (for example, sourness and harshness can be further reduced). In the present invention, the “prtS gene” refers to a gene encoding a cell wall-associated serine protease that degrades casein. Moreover, in the present invention, whether or not S. thermophilus has the prtS gene can be determined by, for example, whether or not the desired PCR product is obtained by amplifying a portion of the prtS gene using the following primers prepared from a highly conserved sequence of the pats gene by the method described in the Examples below.

Such S. thermophilus prtS(+) preferably includes S. thermophilus identified by accession number NITE BP-02875. The S. thermophilus identified by accession number NITE BP-02875 is S. thermophilus prtS(+) derived from Japanese raw milk.

The S. thermophilus identified by accession number NITE BP-02875 has been deposited at the depositary institution with (1) identification label: Streptococcus thermophilus OLS4496, (2) accession number: NITE BP-02875, (3) date of accession: Feb. 5, 2019, and (4) depositary institution: National Institute of Technology and Evaluation Patent Microorganisms Depositary (NPMD) (postal code 292-0818, Room 122, 2-5-8 Kazusa Kamatari, Kisarazu City, Chiba Prefecture). Note that the S. thermophilus identified by accession number NITE BP-02875 may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

### Lactic Acid Bacteria of the Lactobacillaceae

The lactic acid bacterium of the Lactobacillaceae according to the present invention is a lactic acid bacterium belonging to the Lactobacillaceae other than Lactobacillus delbrueckii.

In the present specification, unless otherwise specified, lactic acid bacteria of the Lactobacillaceae are classified into each genus based on Zheng et al., Int. J. Syst. Evol. Microbiol. 2020; 70; 2782-2858. That is, the names of lactic acid bacteria of the Lactobacillaceae in the present specification are names classified based on the above paper, unless otherwise specified. On the other hand, in the present specification, the “old classification” is the classification prior to the publication of the above paper, and if the “old classification name” is mentioned, it indicates that the name is based on the classification prior to the publication of the above paper.

The above Lactobacillus delbrueckii (in the present specification, sometimes referred to as “L. delbrueckii”) is a bacterium classified as the delbrueckii species in the genus Lactobacillus, and its known subspecies include Lactobacillus delbrueckii subsp. Bulgaricus (Bulgarian bacterium), Lactobacillus delbrueckii subsp. delbrueckii, Lactobacillus delbrueckii subsp. lactis, Lactobacillus delbrueckii subsp. indicus, Lactobacillus delbrueckii subsp. sunkii, and Lactobacillus delbrueckii subsp. jakobsenii, and L. delbrueckii according to the present invention also includes subspecies such as these.

The lactic acid bacterium of the Lactobacillaceae according to the present invention is not particularly limited as long as it is other than the above L. delbrueckii, and may be used singly or in combination of two or more types. In the present invention, by using the above S. thermophilus in combination, it is possible to significantly shorten the time until fermentation completion even when using such lactic acid bacteria of the Lactobacillaceae other than L. delbrueckii, and it is possible to obtain fermented milk having an excellent flavor characteristic (for example, mild flavor with low sourness but with milkiness and sweetness) differentiated from conventional fermented milk and fermented milk using L. delbrueckii (preferably yogurt).

Examples of the lactic acid bacteria of the Lactobacillaceae according to the present invention include lactic acid bacteria in the genus Lactobacillus (excluding L. delbrueckii), lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

More specific examples include Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, including Lactobacillus kefiranofaciens subsp. kefiranofaciens, Lacticaseibacillus paracasei, including Lacticaseibacillus paracasei subsp. paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc cremoris, Leuconostoc mesenteroides, including Leuconostoc mesenteroides subsp. mesenteroides, and Leuconostoc pseudomesenteroides.

Furthermore, among these, the lactic acid bacterium of the Lactobacillaceae according to the present invention is preferably at least one selected from the group consisting of lactic acid bacteria in the genus Lactobacillus (excluding L. delbrueckii), lactic acid bacteria in the genus Lacticaseibacillus, and lactic acid bacteria in the genus Lactiplantibacillus, from the viewpoint that the time until fermentation completion can be sufficiently shortened, and the flavor of fermented milk tends to be further improved (for example, sourness and harshness can be further reduced), if combined with S. thermophilus. All of these lactic acid bacteria of the Lactobacillaceae are lactic acid bacteria belonging to the genus Lactobacillus, lactic acid bacteria belonging to the genus Pediococcus, or lactic acid bacteria belonging to the genus Leuconostoc in the old classification, but are more preferably lactic acid bacteria belonging to the genus Lactobacillus in the old classification.

The lactic acid bacterium in the genus Lactobacillus (excluding L. delbrueckii) is more preferably at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, and Lactiplantibacillus plantarum. Further preferable examples include Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens.

Further preferable examples of such lactic acid bacteria in the genus Lactobacillus include the Lactobacillus gasseri JCM 1131^{T} strain, the Lactobacillus gasseri P2001801 strain, the Lactobacillus gasseri P2001802 strain, the Lactobacillus helveticus JCM 1120^{T} strain, the Lactobacillus helveticus P2001803 strain, the Lactobacillus helveticus P2001804 strain, the Lactobacillus acidophilus JCM 1132^{T} strain, the Lactobacillus amylovorus JCM 1126^{T} strain, the Lactobacillus crispatus JCM 1185^{T} strain, the Lactobacillus johnsonii JCM 2012^{T} strain, the Lactobacillus kefiranofaciens subsp. kefiranofaciens JCM 6985^{T} strain, and the Lactobacillus paragasseri JCM 5343^{T} strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

Note that in the present specification, strains whose bacterial strain numbers are indicated by JCM are strains available from RIKEN BioResource Research Center Microbe Division (http://jcm.brc.riken.jp/ja/), strains whose strain numbers are indicated by NBRC are strains available from National Institute of Technology and Evaluation Biological Resource Center (http://www.nite.go.jp/nbrc/), and strains whose strain numbers are indicated by NCIMB are strains available from the United Kingdom National Culture Collection, the National Collection of Industrial, Food and Marine Bacteria. In addition, in the present specification, strains whose bacterial strain numbers start with P20018 are bacterial strains kept at the Meiji Innovation Center of Meiji Co., Ltd. (postal code 192-0919, 1-29-1 Nanakuni, Hachioji City, Tokyo, Japan).

More preferable examples of lactic acid bacteria in the genus Lacticaseibacillus include Lacticaseibacillus paracasei (old classification name: Lactobacillus paracasei), Lacticaseibacillus rhamnosus (old classification name: Lactobacillus rhamnosus), and Lacticaseibacillus casei (old classification name: Lactobacillus casei).

Further preferable examples of such lactic acid bacteria in the genus Lacticaseibacillus include the Lacticaseibacillus paracasei subsp. paracasei NBRC 15889^{T} (old classification name: Lactobacillus paracasei subsp. paracasei NBRC 15889^{T}) strain, the Lacticaseibacillus paracasei P2001805 (old classification name: Lactobacillus paracasei P2001805) strain, the Lacticaseibacillus paracasei NITE BP-02244 (old classification name: Lactobacillus paracasei NITE BP-02244) strain, the Lacticaseibacillus rhamnosus JCM 1136^{T} (old classification name: Lactobacillus rhamnosus JCM 1136T) strain, the Lacticaseibacillus rhamnosus P2001808 (old classification name: Lactobacillus rhamnosus P2001808) strain, the Lacticaseibacillus rhamnosus P2001809 (old classification name: Lactobacillus rhamnosus P2001809) strain, the Lacticaseibacillus casei JCM 1134^{T} (old classification name: Lactobacillus casei JCM 1134^{T}) strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

Note that Lacticaseibacillus paracasei NITE BP-02244 is Lacticaseibacillus paracasei identified by accession number NITE BP-02244 and has been deposited at the depositary institution with (1) identification label: Lactobacillus paracasei subsp. paracasei OLL204220, (2) accession number: NITE BP-02244, (3) date of accession: Apr. 25, 2016, and (4) depositary institution: National Institute of Technology and Evaluation Patent Microorganisms Depositary (NPMD) (postal code 292-0818, Room 122, 2-5-8 Kazusa Kamatari, Kisarazu City, Chiba Prefecture).

More preferable examples of lactic acid bacteria in the genus Lactiplantibacillus include Lactiplantibacillus plantarum (old classification name: Lactobacillus plantarum), Lactiplantibacillus paraplantarum (old classification name: Lactobacillus paraplantarum), and Lactiplantibacillus pentosus (old classification name: Lactobacillus pentosus).

Further preferable examples of such lactic acid bacteria in the genus Lactiplantibacillus include the Lactiplantibacillus plantarum NCIMB 11974^{T} (old classification name: Lactobacillus plantarum NCIMB 11974^{T}) strain, the Lactiplantibacilius plantarum P2001806 (old classification name: Lactobacillus plantarum P2001806) strain, the Lactiplantibacillus plantarum P2001807 (old classification name: Lactobacillus plantarum P2001807′) strain, the Lactiplantibacillus paraplantarum NCIMB 13579^{T} strain, and the Lactiplantibacillus pentosus NCIMB 8026^{T} strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Latilactobacillus include Latilactobacillus sakei (old classification name: Lactobacillus sakei).

Further preferable examples of such lactic acid bacteria in the genus Latilactobacillus include the Latilactobacillus sakei 0-CM 1157^{T} (old classification name Lactobacillus sakei JCM 1157^{T}) strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Liquorilactobacillus include Liquorilactobacillus cacaonum (old classification name: Lactobacillus cacaonum) and Liquorilactobacillus satsumensis (old classification name: Lactobacillus satsumenais).

Further preferable examples of such lactic acid bacteria in the genus Liquorilactobacillus include the Liquorilactobacillus cacaonum P2001810 (old classification name: Lactobacillus cacaonum P2001810) strain and Liquorilactobacillus satsumensis JCM 12392^{T} (old classification name: Lactobacillus satsumensis JCM 12392^{T}). Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Ligilactobacillus include Ligilactobacillus salivarius (old classification name: Lactobacillus salivarius).

Further preferable examples of such lactic acid bacteria in the genus Ligilactobacillus include the Ligilactobacillus salivarius JCM 1231^{T} (old classification name: Lactobacillus salivarius JCM 1231^{T}) strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Limosilactobacillus include Limosilactobacillus fermentum (old classification name: Lactobacillus fermentum) and Limosilactobacillus reuteri (old classification name: Lactobacillus reuteri).

Further preferable examples of such lactic acid bacteria in the genus Limosilactobacillus include the Limosilactobacillus fermentum JCM 1173^{T} (old classification name: Lactobacillus fermentum JCM 1173^{T}) strain and the Limosilactobacillis reuteri JCM 1112^{T} (old classification name: Lactobacillus reuteri JCM 1112^{T}) strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Levilactobacillus include Levilactobacillus brevis (old classification name: Lactobacillus brevis) and Levilactobacillus namurensis (old classification name: Lactobacillus namurensis).

Further preferable examples of such lactic acid bacteria in the genus Levilactobacillus include the Levilactobacillus brevis JCM 1059^{T} (old classification name: Lactobacillus brevis JCM 1059^{T}) strain and the Levilactobacillus namurensis NBRC 107158^{T} (old classification name: Lactobacillus namurensis NBRC 107158^{T}) strain. Each of these bacterial strains may be a subcultured strain of the same, strain, or may be an artificial mutant strain, a natural mutant strain, a genetically modified strain, a derivative strain, car the like of the same strain or a subculture strain thereof, as long as the effects of the present invention are not impaired.

More preferable examples of lactic acid bacteria in the genus Lentilactobacillus include Lentilactobacillus buchneri (old classification name: Lactobacillus buchneri), Lentilactobacillus parabuchneri (old classification name: Lactobacillus parabuchneri), and Lentilactobacillus kefiri (old classification name: Lactobacillus kefiri).

Further preferable examples of such lactic acid bacteria in the genus Lentilactobacillus include the Lentilactobacillus buchneri NCIMB 8007^{T} (old classification name: Lactobacillus buchneri NCIMB 8007^{T}) strain, the Lentilactobacillus parabuchneri JCM 12493^{T} (old classification name: Lactobacillus parabuchneri JCM 12493^{T}) strain, and the Lentilactobacillus kefiri JCM 5818^{T} (old classification name: Lactobacillus kefiri JCM 5818^{T}) strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Pediococcus include Pediococcus pentosaceus and Pediococcus acidilactici.

Further preferable examples of such lactic acid bacteria in the genus Pediococcus include the Pediococcus pentosaceus JCM 5890^{T} strain and the Pediococcus acidilactici JCM 8797^{T} strain. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

More preferable examples of lactic acid bacteria in the genus Leuconostoc include Leuconostoc lactis, Leuconostoc mesenteroides, and Leuconostoc pseudomesenteroides.

Further preferable examples of such lactic acid bacteria in the genus Leuconostoc include the Leuconostoc lactis JCM 6123^{T} strain, the Leuconostoc mesenteroides subsp. mesenteroides JCM 6124^{T} strain, and Leuconostoc pseudomesenteroides JCM 9696^{T}. Each of these bacterial strains may be a subcultured strain of the same strain, or may be an artificial mutant strain, a natural mutant strain, a genetically modified strain, a derivative strain, or the like of the same strain or a subculture strain thereof, as long as the effects of the present invention are not impaired.

Among the above, the lactic acid bacterium of the Lactobacillaceae according to the present invention is more preferably at least one selected from the group consisting of Lactobacillus gasseri, Lactobacillus paragasseri, Lactobacillus helveticus, Lacticaseibacillus paracasei, Lacticaseibacillus rhamnosus, and Lactobacillus plantarum.

The lactic acid bacteria starter of the present invention may be a composition containing the above S. thermophilus and lactic acid bacteria of the Lactobacillaceae, or may be a combination containing the above S. thermophilus and lactic acid bacteria of the Lactobacillaceae.

When the lactic acid bacteria starter of the present invention is the composition described above, the composition may be in a liquid state or in a solid state such as a frozen state or a dry powder, and may further contain additional components. Examples of the additional components include cultures such as culture supernatants and medium components (such as milk and whey) after culturing lactic acid bacteria (S. thermophilus, lactic acid bacteria of the Lactobacillaceae); concentrates, dilutions, dried products, frozen products, and the like of the above cultures; fermentation promoting substances (such as formic acid and nucleic acids); and protective agents (saccharides), and may be one of these or a combination of two or more thereof.

In addition, when the lactic acid bacteria starter of the present invention is the composition described above, the ratio of the content of S. thermophilus to the content of lactic acid bacteria of the Lactobacillaceae in the composition (S. thermophilus: lactic acid bacteria of the Lactobacillaceae (excluding L. delbrueckii)) is preferably 1:0.1 to 1:100, more preferably 1:1 to 1:10, in bacteria count (in terms of viable bacteria count, hereinafter the same) ratio. In addition, when the lactic acid bacteria starter of the present invention is the composition described above, the total content of S. thermophilus and lactic acid bacteria of the Lactobacillaceae (excluding L. delbrueckii) in the lactic acid bacteria starter is not particularly limited, but is preferably 0.01 to 100% by mass, more preferably 0.1 to 90% by mass. Further, when the lactic acid bacteria starter of the present invention is the composition described above, the total content of S. thermophilus and lactic acid bacteria of the Lactobacillaceae (excluding L. delbrueckii) in the lactic acid bacteria starter is not particularly limited, but is preferably 1×10⁷ cfu/g or more, more preferably 1×10⁷ to 1×10¹¹ cfu/g, and further preferably 1×10⁸ to 1×10¹⁰ cfu/g.

When the lactic acid bacteria starter of the present invention is the combination described above, examples of the combination include a combination of a first lactic acid bacteria composition containing S. thermophilus and a second lactic acid bacteria composition containing lactic acid bacteria of the Lactobacillaceae (excluding L. delbrueckii), which can be a kit containing the first lactic acid bacteria composition and the second lactic acid bacteria composition. In this case, the first lactic acid bacteria composition and the second lactic acid bacteria composition may be each independently in a liquid state or in a solid state such as a frozen state or a dry powder, and may be composed only of the corresponding lactic acid bacteria or may further contain additional components. Examples of the additional components include the same as the additional components listed when the lactic acid bacteria starter is a composition.

In addition, the content of S. thermophilus in the first lactic acid bacteria composition and the content of lactic acid bacteria of the Lactobacillaceae (excluding L. delbrueckii) in the second lactic acid bacteria composition are not particularly limited, but are each independently preferably 0.01 to 100% by mass, and more preferably 0.1 to 90% by mass. Moreover, the content of S. thermophilus in the first lactic acid bacteria composition and the content of lactic acid bacteria of the Lactobacillaceae (excluding L. delbrueckii) in the second lactic acid bacteria composition are not particularly limited, but are each independently preferably 1×10⁷ cfu/g or more, more preferably 1×10⁷ to 1×10¹¹ cfu/g, and further preferably 1×10⁸ to 1×10¹⁰ cfu/g. The first lactic acid bacteria composition and the second lactic acid bacteria composition are used such that the ratio of S. thermophilus to lactic acid bacteria of the Lactobacillaceae according to the present invention (S. thermophilus: lactic acid bacteria of the Lactobacillaceae (excluding L. delbrueckii)) in bacteria count ratio is preferably 1:0.1 to 1:100, more preferably 1:1 to 1:10.

In addition, when the lactic acid bacteria starter of the present invention is the above kit, the kit may further include additives for producing fermented milk (such a the above fermentation promoting substances and protective agents), container, instructions for use of the lactic acid bacteria starter, and the like.

### Method for Producing Fermented Milk

The method for producing fermented milk of the present invention includes a fermentation step of adding the lactic acid bacteria starter of the present invention to a milk preparation solution containing raw material milk and fermenting the mixture to obtain fermented milk. Specifically, it includes a fermentation step of adding S. thermophilus according to the present invention and lactic acid bacteria of the Lactobacillaceae other than L. delbrueckii to the milk preparation solution containing raw material milk and fermenting the mixture to obtain fermented milk. By fermenting raw material milk using the combination of S. thermophilus according to the present invention and lactic acid bacteria of the Lactobacillaceae, the production method of the present invention can sufficiently shorten the time until fermentation completion, and makes it possible to obtain fermented milk having a flavor characteristic differentiated from conventional fermented milk and fermented milk using L. delbrueckii (preferably yogurt), “mild flavor with low sourness but with milkiness and sweetness”.

### Milk Preparation Solution

The milk preparation solution according to the present invention contains raw material milk. The raw material milk preferably contains lactose, and examples thereof include raw milk (such as milk of cow, water buffalo, sheep, goat, and the like), pasteurized milk, whole milk, skim milk, whey, and processed products thereof (such as whole milk powder, full-fat concentrated milk, skimmed milk powder, skimmed concentrated milk, condensed milk, whey powder, buttermilk, butter, cream, cheese, whey protein concentrate (WPC), whey protein isolate (WPI), α-lactalbumin (α-La), and β-lactoglobulin (β-Lg)), and may be one of these or a mixture of two or more thereof.

The milk preparation solution according to the present invention may be composed only of the above raw material milk, or may be an aqueous solution, diluted solution, or concentrated solution of the above raw material milk, or may further contain additional components it addition to the above raw material milk, if necessary. Examples of the additional components include water; foods, food ingredients, and food additives such as soymilk, saccharides such as sugar, sweeteners, flavors, fruit juices, fruit pulps, vitamins, minerals, oils and/or fats, ceramides, collagen, milk phospholipids, and polyphenols; stabilizers, thickeners, and gelling agents such as pectin, soy polysaccharides, CMC (carboxymethylcellulose), agar, gelatin, carrageenan, and gums, and may be one of these or a mixture of two or more thereof. The milk preparation solution can be prepared by mixing the above components, while optionally with heating and/or optionally with homogenizing. In addition, as the milk preparation solution, heat-sterilized one can also be used.

### Fermentation

The fermentation step of adding the lactic acid bacteria starter of the present, invention (that is, the combination of S. thermophilus according to the present invention and lactic acid bacteria of the Lactobacillaceae) to the milk preparation solution and fermenting the mixture can appropriately employ conventionally known method, and is not particularly limited. The S. thermophilus according to the present invention and lactic acid bacteria of the Lactobacillaceae may be added to the milk preparation solution after being mixed, or may be added simultaneously or separately. That is, the fermentation starter of the present invention may be added to the milk preparation solution as a composition containing S. thermophilus according to the present invention and lactic acid bacteria of the Lactobacillaceae, or the first lactic acid bacteria composition containing S. thermophilus above and the second lactic acid bacteria composition containing lactic acid bacteria of the Lactobacillaceae may be added simultaneously or separately.

The amount of the fermentation starter (that is, the combination of S. thermophilus according to the present invention and lactic acid bacteria of the Lactobacillaceae) added can be appropriately set according to the amount to be added employed in conventionally known methods for producing fermented milk, and is, for example, preferably 1×10⁷ and 5×10⁹ cfu/mL, more preferably 1×10and 2×10⁹ cfu/mL, based on the volume of the milk preparation solution, in terms of lactic acid bacteria count (total bacteria count of S. thermophilus and lactic acid bacteria of the Lactobacillaceae (excluding L. delbrueckii)).

In addition, the ratio of S. thermophilus to lactic acid bacteria of the Lactobacillaceae (S. thermophilus: lactic acid bacteria of the Lactobacillaceae (excluding L. delbrueckii)) added to the milk preparation solution is preferably 1:0.1 to 1:100, more preferably 1:1 to 1:10.

The fermentation conditions are not particularly limited, and can be appropriately selected depending on the growth conditions of S. thermophilus and lactic acid bacteria of the Lactobacillaceae to be added, the amount of the milk preparation solution, and the like. For example, under aerobic or anaerobic conditions at a temperature of 35 to 45° C. and more preferably at a temperature of 38 to 43° C., the mixture is allowed to stand or stirred (preferably stand) for usually 3 to 24 hours, more preferably 3 to 8 hours, and further preferably 4 to 6 hours until the pH of the milk preparation solution added with S. thermophilus according to the present invention and lactic acid bacteria of the Lactobacillaceae is 4.5. or less, more preferably 4.0 to 4.5. According to the present invention, it is possible to sufficiently shorten the time required for fermentation even when using the lactic acid bacteria of the Lactobacillaceae according to the present invention other than L. delbrueckii. In addition, as the anaerobic conditions, for example, fermentation under nitrogen aeration conditions can be employed.

The fermented milk of the present invention can be obtained by the above fermentation. The fermented product after the fermentation step can be used as the fermented milk of the present invention as it is or by concentrating, diluting, drying, freezing, or the like as necessary. Also, the fermented milk of the present invention may be obtained by crushing or heat-treating the lactic acid bacteria in the fermented product, or by concentrating, diluting, drying, freezing, or the like as necessary. Therefore, the method for producing fermented milk of the present invention may further include these steps (such as concentration step, dilution step, drying step, freezing step, crushing step, and heat treatment step).

### Fermented Milk

The fermented milk of the present invention contains S. thermophilus according to the present invention and lactic acid bacteria of the Lactobacillaceae described above, and can be obtained by the above method for producing fermented milk of the present invention.

The fermented milk of the present invention is not particularly limited, and examples thereof include fermented milk that satisfies the standards for “fermented milk” according to the Ministerial Ordinance on Milk and Milk Products Concerning Compositional Standards, etc. (Ministerial Ordinance on Milk and Milk Products) issued by the Ministry of Health, Labor and Welfare of Japan (more specifically, the content of milk solids-not-fat is 8.0% or more, and the lactic acid bacteria count or yeast count (preferably the lactic acid bacteria count (more preferably the total count of S. thermophilus and lactic acid bacteria of the Lactobacillaceae, hereinafter the same)) is 10 million/mL or more). In addition, the fermented milk of the present invention also includes those that satisfy the standards for “milk products and fermented milk drinks” (more specifically, the content of milk solids-not-fat is 3.0% or more, and the lactic acid bacteria count or yeast count (preferably the lactic acid bacteria count) is 10 million/min or more); those that satisfy the standards for “fermented milk drinks” (more specifically, the content of milk solids-not-fat is 3.0% or more, and the lactic acid bacteria count or yeast count (preferably the lactic acid bacteria count) is 1 million/mL or more), according to the above Ministerial Ordinance on Milk and Milk Products. Note that the milk solids-not-fat refers to the remaining components (mainly such as proteins, lactose, and minerals) obtained by subtracting the fat content from the total milk solids, and if the fermented milk is to be pasteurized, the lactic acid bacteria and yeast count are measured by the test method specified in the above Ministerial Ordinance on Milk and Milk Products before the pasteurization.

The fermented milk of the present invention may be a fermented product after the fermentation step or may be obtained by pasteurizing the fermented product (such as pulverization or heat treatment), or may be obtained by, for example, concentrating, diluting, drying, or freezing them. Note that in the present invention, when the fermented milk is pasteurized, the lactic acid bacteria count (preferably the total count of S. thermophilus and lactic acid bacteria of the Lactobacillaceae (excluding L. delbrueckii)) in the fermented milk is in terms of viable bacteria count. The lactic acid bacteria contained in the fermented milk of the present invention include not only viable cell but also dead cell, as well as crushed products and heat-treated products of lactic acid bacteria, and concentrates, dilutions, dried products, and frozen products thereof. The lactic acid bacteria contained in the fermented milk of the present invention preferably include at least viable bacteria.

The fermented milk of the present invention may contain, as lactic acid bacteria, lactic acid bacteria other than the S. thermophilus according to the present invention and lactic acid bacteria of the Lactobacillaceae, and may further contain yeast, as long as the effects of the present invention are not impaired. These other lactic acid bacteria and yeasts include lactic acid bacteria and yeasts that are conventionally known to be contained in fermented milk.

In addition, the fermented milk of the present invention may further contain various components that can be contained in food and drink. Such components are not particularly limited, and examples thereof include water, saccharides, sugar alcohols, minerals, vitamins, proteins, peptides, amino acids, organic acids, pH adjusters, starches and modified starches, dietary fibers, fruits and vegetables and processed products thereof, animal and plant crude drug extracts, naturally-derived polymers (such as collagen, hyaluronic acid, and chondroitin), oils and/or fats, thickeners, emulsifiers, solvents, surfactants, gelling agents, stabilizers, buffers, suspending agents, thickening agents, excipients, disintegrators, binders, flow agents, preservatives, colorants, flavors, corrigents, and sweeteners. One of these may be contained alone, or two or more thereof ray be contained in combination.

Such fermented milk is particularly preferably yogurt. Specific examples of the yogurt include set type yogurt (solid fermented milk) such as plain yogurt, soft-type yogurt (pasty fermented milk), and drink type yogurt (liquid fermented milk), and frozen yogurt using these ingredients may also be used. In addition, the fermented milk of the present invention can also be used as an ingredient for fermented milk such as cheese, fermented cream, fermented butter, and kefir.

### Method for Promoting Fermentation of Fermented Milk, and Method for Improving Flavor of Fermented Milk

By fermenting raw material milk using the combination of S. thermophilus according to the present invention and lactic acid bacteria of the Lactobacillaceae, it is possible to sufficiently shorten the time until fermentation completion fora the production of fermented milk, and makes it possible to obtain fermented milk having a flavor characteristic differentiated from conventional fermented milk, “mild flavor with low sourness but with milkiness and sweetness”. Therefore, the present invention provides a method for promoting fermentation of fermented milk as well as a method for improving flavor of fermented milk that include a fermentation step of adding the lactic acid bacteria starter of the present invention (that is, the combination of S. thermophilus according to the present invention and lactic acid bacteria of the Lactobacillaceae) to the milk preparation solution containing raw material milk and fermenting the mixture to obtain fermented milk. The fermentation step is as described in Method for Producing Fermented Milk of the present invention.

Compared to the fermentation with single use of the lactic acid bacteria of the Lactobacillaceae according to the present invention, the method for promoting fermentation of fermented milk of the present invention can significantly shorten the time until fermentation completion (in the present specification, preferably the time until pH becomes 4.5 or less). For example, if the time until fermentation completion in the case of fermentation with single use of the lactic acid bacteria of the Lactobacillaceae according to the present invention is set to 1, the time until fermentation completion can be shortened to 0.7 or less, more preferably 0.1 to 0.7, further preferably 0.1 to 0.5, and particularly preferably 0.1 to 0.3.

In addition, compared to the fermentation with single use of the lactic acid bacteria of the Lactobacillaceae according to the present invention, the method for improving flavor of fermented milk of the present invention can further improve the flavor of the resulting fermented milk to a better mild flavor with low sourness but with milkiness and sweetness, even compared to fermented milk (preferably yogurt) using L. delbrueckii (preferably yogurt).

Furthermore, in the case of using S. thermophilus carrying the prtS gene as the S. thermophilus according to the present invention, there is a tendency to further shorten the time until fermentation completion, and it is possible to improve the flavor of the resulting fermented milk to a flavor with less sourness and harshness.

### EXAMPLES

The present invention will be described in more detail below based on Examples and Comparative Examples, but the present invention is not limited to the following Examples.

### Test Example 1

### S. thermophilus

As S. thermophilus, the bacterial strains shown in Table 1 below were used. In Table 1,
S. thermophilus prtS(−): “S. thermophilus 1131”, isolated from Meiji Bulgaria Yogurt LB81 (manufactured by Meiji Co., Ltd.), without the prtS gene; and
S. thermophilus prtS(+): “S. thermophilus NITE BP-02875”, S. thermophilus identified by accession number NITE BP-02875 (deposited at the depositary institution with (1) identification label: Streptococcus thermophilus OLS4496, (2) accession number: NITE BP-02875, (3) date of accession: Feb. 5, 2019, and (4) depositary institution: National Institute of Technology and Evaluation Patent Microorganisms Depositary (NPMD) (postal code 292-0818, Room 122, 2-5-8 Kazusa Kamatari, Kisarazu City, Chiba Prefecture)), with the prtS gene.

Note that the presence or absence of the prtS gene in each S. thermophilus was confirmed by the following method. Specifically, first, the prtS gene sequences of 5 strains of thermophilus bacteria with known genome sequences were obtained from the NCBI database, and the highly conserved sequences were used to prepare primers (forward primer: SEQ ID NO: 1 and reverse primer: SEQ ID NO: 2). In addition, InstaGene Matrix (manufactured by BioRad) was used to extract genomic DNA from the M17 culture medium of each bacterial strain. 0.5 μL of extracted genomic DNA (template), 1 μL each of the prepared primers (5 μM), 0.1 μL of Phusion high fidelity DNA polymerase, 2 μL of 5×HF buffer, 0.8 μL of 2.5 mM dNTP, and 4.6 μL of ultrapure water were mixed (total 10 μL), and PCR was performed under the following conditions: at 98° C. for 30 seconds; 30 cycles of 98° C. for 5 seconds, 63° C. for 20 seconds, and 72′C for 20 seconds; and 72° C. for 5 minutes; and left at 4° C. The resulting PCR products were subjected to agarose gel electrophoresis, and bacterial strains confirmed to have a band at 684 by were determined to have the prtS gene, and bacterial strains not confirmed to have the band were determined not to have the prtS gene.

### Lactic Acid Bacteria of the Lactobacillaceae

As lactic acid bacteria of the Lactobacillaceae, the bacterial strains of the bacterial species shown in Table 1 below were used. In Table 1,
Lactobacillus delbrueckii 2038: L. delbrueckii isolated from Meiji Bulgaria Yogurt LB81 (manufactured by Meiji Co., Ltd.);
Lactobacillus delbrueckii NITE BP 76: L. delbrueckii identified by accession number NITE BP-76 (deposited at the depositary institution with (1) identification label Lactobacillus delbrueckii subspecies bulgaricus O LL1255, (2) accession number NITE BP-76, (3) date of accession (date of original deposit) Feb. 10, 2005, and (4) depositary institution: National Institute of Technology and Evaluation Patent Microorganisms Depositary (NPMD) (postal code 292-0819, 2-5-8 Kazusa Kamatari, Kisarazu City, Chiba Prefecture));
Lactobacillus delbrueckii NITE BP-02874: L. delbrueckii identified by accession number NITE BP-02874 (deposited at the depositary institution with (1) identification Lactobacillus delbrueckii OLL204989, (2) accession number: NITE BP-02874, (3) date of accession: Feb. 5, 2019, and (4) depositary institution National Institute of Technology and Evaluation Patent Microorganisms Depositary (NPMD) (postal code 292-0818, Room 122, 2-5-8 Kazusa Kamatari, Kisarazu City, Chiba Prefecture)); and
Lacticaseibacillus paracasei NITE BP-02244: Lacticaseibacillus paracasei identified by accession number NITE BP-02244 (deposited at the depositary institution with (1) identification label: Lactobacillus paracasei subsp. paracasei OLL204220, (2) accession number: NITE BP-02244, (3) date of accession: Apr. 25, 2016, (4) depositary institution: National Institute of Technology and Evaluation Patent Microorganisms Depositary (NPMD) (postal code 292-0818, Room 122, 2-5-8 Kazusa Kamatari, Kisarazu City, Chiba Prefecture)).

In addition, in Table 1, strains whose bacterial strain numbers are indicated by JCM were obtained from RIKEN BioResource Research Center Microbe Division (http://jcm.brc.riken.jp/ja/), strains whose strain numbers are indicated by NBRC were obtained from National Institute of Technology and Evaluation Biological Resource Center (http://www.nite.go.jp/nbrc/), and strains whose strain numbers are indicated by NCIMB were obtained from the United Kingdom National Culture Collection, the National Collection of Industrial, Food and Marine Bacteria. Moreover, in Table 1, strains whose bacterial strain numbers start with P20018 are bacterial strains kept at the Meiji Innovation Center of Meiji Co., Ltd. (postal code 192-0919, 1-29-1 Nanakuni, Hachioji City, Tokyo, Japan).

### Fermentation Time Measurement

The above S. thermophilus and lactic acid bacteria of the Lactobacillaceae were used in the combinations shown in Table 1 below (Examples: R4 to R18, R22 to R36, Comparative Examples: R1 to R3, R19 to R21, L1 to L18) to measure the time required for fermentation.

TABLE 1

| | | S. thermophilus | | |
|---|---|---|---|---|
| Lactic Acid Bacteria of the Lactobacillaseae | | | prts (+) | |
| | Bacterial Species | prts (−) | NITE | |
| Bacterial Species Name | Number | 1131 | BP-02875 | None |
| Lactobacillus delbrueckii | 2038 | R1 | R19 | L1 |
| | NITE BP-76 | R2 | R20 | L2 |
| | NITE BP-02874 | R3 | R21 | L3 |
| Lactobacillus gasseri | JCM 1131^{T} | R4 | R22 | L4 |
| | P2001801 | R5 | R23 | L5 |
| | P2001802 | R6 | R24 | L6 |
| Lactobacillus helveticus | JCM 1120^{T} | R7 | R25 | L7 |
| | P2001803 | R8 | R26 | L8 |
| | P2001804 | R9 | R27 | L9 |
| Lacticaseibacillus paracasei | NBRC 15889^{T} | R10 | R28 | L10 |
| | P2001805 | R11 | R29 | L11 |
| | NITE BP-02244 | R12 | R30 | L12 |
| Lactiplantibacillus plantarum | NCIMB 11974^{T} | R13 | R31 | L13 |
| | P2001806 | R14 | R32 | L14 |
| | P2001807 | R15 | R33 | L15 |
| Lacticaseibacillus rhamnosus | JCM 1136^{T} | R16 | R34 | L16 |
| | P2001808 | R17 | R35 | L17 |
| | P2001809 | R18 | R36 | L18 |

| | |
|---|---|
| None | Blank |

For fermentation, 0.5% (0.5 mL/100 mL, hereinafter the same) of each bacterial strain or a combination thereof (S. thermophilus: lactic acid bacteria of the Lactobacillaceae=about 1:1 (bacteria count)) was added to commercially available milk so that the amount of each bacterial strain was 0.5% (1% in total), which was fermented at 43° C. for 24 hours (aerobic, stationary culture) to obtain each fermented milk. The time from the addition of each bacterial strain until the pH became 4.5 or less was measured and used as the fermentation time required for fermentation. If the pH did not reach 4.5 within 24 hours after the addition of bacterial strain, the fermentation was terminated with the fermentation time set to 24 hours (1440 minutes).

As a result, of fermentation time measurement, it was confirmed that when S. thermophilus was used (R1 to R36), the fermentation time was shortened compared to when fermentation was performed using lactic acid bacteria of the Lactobacillaceae alone without using S. thermophilus (L1 to L18). In particular, when Lactobacillus helveticus and Lacticaseibacillus rhamnosus were used (R7 to R9, R16 to R18, R25 to R27, R34 to R36), the fermentation time was sufficiently shortened regardless of whether S. thermophilus carried the prtS gene, and as a result of the t-test, the p-value was less than 0.001 compared to the case of lactic acid bacteria of the Lactobacillaceae alone (L7 to L9, L16 to L18). The same was true when L. delbrueckii was used (R1 to R3, R19 to R21). Table 2 below shows fermentation time (min) when using S. thermophilus carrying the prtS gene (R19 to R36) and when fermenting with lactic acid bacteria of the Lactobacillaceae alone (L1 to L13, L16 to L18).

TABLE 2

| Combination | Fermentation Time | Combination | Fermentation Time |
|---|---|---|---|
| No. | [min] | No. | [min] |
| R19 | 415 | L1 | 1440 |
| R20 | 345 | L2 | 1440 |
| R21 | 345 | L3 | 1440 |
| R22 | 505 | L4 | 1440 |
| R23 | 485 | L5 | 1440 |
| R24 | 575 | L6 | 1440 |
| R25 | 495 | L7 | 1325 |
| R26 | 615 | L8 | 1440 |
| R27 | 625 | L9 | 1440 |
| R28 | 640 | L10 | 1440 |
| R29 | 535 | L11 | 1440 |
| R30 | 560 | L12 | 1440 |
| R31 | 660 | L13 | 1440 |
| R32 | 640 | L14 | — |
| R33 | 605 | L15 | 1440 |
| R34 | 435 | L16 | 1175 |
| R35 | 440 | L17 | 1440 |
| R36 | 455 | L18 | 1440 |
| Average | 520.8 | Average | 1417.6 |
| SD | 100.8 | SD | 68.4 |

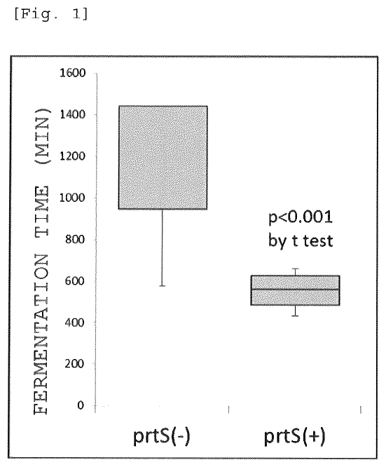

In addition, FIG. 1 shows fermentation time (mean values, min) when using S. thermophilus not carrying the brt gene (prtS(−), R4 to R18 (n=15)) and when using S. thermophilus carrying the puts gene (prtS(+) R22 to R36 (n=15)) for the lactic acid bacteria of the Lactobacillaceae other than L. delbrueckii. As a result of the t-test, the results for prtS(+) had p-values of less than 0.001 with respect to the results for prtS(−). From the above, it was confirmed that prtS(+) shortened the fermentation time more significantly than prtS(−).

### Evaluation of Fermented Milk

### Analysis of Water-Soluble Components

In the above fermentation time measurement, the water-soluble components in the fermented milk obtained using the bacterial strains of the combinations shown in Table 1 were analyzed by CE-TOFMS (capillary electrophoresis time-of-flight mass spectrometer) according to the following method (Human Metabolome Technologies, Inc.).

### Pretreatment

To 900 μL of methanol solution prepared so that the concentration of the internal standard substance was 10 μM, 100 μL of fermented milk was added and stirred. To this, 1,000 μL of chloroform and 400 μL of ultrapure water were added, stirred, and centrifuged (2,300×g, 4° C., 5 minutes). After centrifugation, 400 μL of the aqueous layer was transferred to an ultrafiltration tube (Ultrafree MC PLHCC, HMT, centrifugal filter unit 5 kDa). This was centrifuged (9,100×g, 4° C., 120 minutes) and subjected to ultrafiltration. The filtrate was dried and dissolved again in 50 μL of ultrapure water for measurement.

### Measurement

Measurements in cation mode and anion mode were performed under the conditions shown in Table. 3 below.

TABLE 3
Cationic Metabolite (Cation Mode)

| | |
|---|---|
| Device | Agilent CE-TOFMS system (Agilent Technologies, Inc. ) |
| | Capillary: Fused silica capillary i.d. 50 μm × 80 cm |
| Measurement Condition | Run buffer: Cation Buffer Solution (p/n: H3301-1001) |
| | Rinse buffer: Cation Buffer Solution (p/n: H3301-1001) |
| | Sample injection: Pressure injection 50 mbar, 10 sec |
| | CE voltage: Positive, 27 kV |
| | MS ionization: ESI Positive |
| | MS capillary voltage: 4,000 V |
| | MS scan range: m/z 50-1,000 |
| | Sheath liquid: HMT Sheath Liquid (p/n: H3301-1020) |

Anionic Metabolite (Anion Mode)

Agilent CE-TOFMS system (Agilent Technologies, Inc.)
Capillary: Fused silica capillary i.d. 50 μm × 80 cm
Run buffer: Anion Buffer Solution (p/n: H3302-1021)
Rinse buffer: Anion Buffer Solution (p/n: H3302-1021)
Sample injection: Pressure injection 50 mbar, 25 sec
CE voltage: Positive, 30 kV
MS ionization: ESI Negative
MS capillary voltage: 3,500 V
MS scan range: m/z 50-1,000
Sheath liquid: HMT Sheath Liquid (p/n: H3301-1020)

### Data Processing

For the peaks detected by CE-TOFMS, using the automatic integration software MasterHands ver. 2.17.1.11 (developed by Keio University), peaks with a signal/noise (S/N) ratio of 3 or more were automatically extracted to obtain a mass-to-charge ratio (m/z), peak area value, and migration time (MT). The obtained peak area values were converted to relative area values using the following formula:

Relative area value=(area value of target peak)/(area value of internal standard substance×sample amount).

In addition, since these datasets contain adduct ions such as Na⁺ and K⁺ and fragment ions such as dehydration and deammoniation, these molecular weight-related ions were deleted. However, since there are also substance-specific adducts and fragments, it was impossible to examine all of them. The peaks examined were collated and aligned between samples based on the values of m/z and MT.

### Search for Candidate Metabolites

The detected peaks were collated and searched with all substances registered in the HMT metabolite library and the Known-Unknown library (Human Metabolome Technologies, Inc.) based on the values of m/z and MT. The permissible error for searching was ±0.5 min for MT and ±10 ppm for m/z (mass error (ppm)=(measured value−theoretical value)×10⁶/measured value). When the Candidates could not be narrowed down and the same candidate metabolite was assigned to multiple peaks, branch numbers were assigned.

### Quantitative Determination of Target Metabolite Compounds

Analysis on the target metabolite compounds was performed. For the calibration curve, the peak area corrected with the internal standard substance was used, and the concentration of each substance was calculated as a one-point calibration of 100 μM (internal standard substance: 200 μM).

### Analysis of Aroma Compounds

In the above fermentation time measurement, the aroma compounds in the fermented milk obtained using the bacterial strains of the combinations described in Table 1 were analyzed by GC/MS (gas chromatography mass spectrometry) method using dynamic headspace and headspace solid-phase microextraction methods as sample pretreatments, according to the following method.

### Analysis Method

### Sample:

To a 20 mL vial, 5 g of fermented milk, 5 g of 1 mol/L-phosphate buffer (pH 6:98), and methyl isobutyl ketone and cyclooctanol as internal standards were added and sealed.

### Dynamic Headspace Collector (Manufactured by Gerstel Inc.):

While keeping the above vial at 25° C., the headspace was replaced with 10 mL of nitrogen gas, and the aroma compounds in the nitrogen gas were collected to an adsorbent (TENAX-TA). Then, the adsorbent was thermally desorbed under the conditions shown in Table 4 below and introduced into GC/MS for analysis. The mass spectra of the detected peaks were collated with the NIST mass spectral library to qualify the detected compounds. Furthermore, the peaks were integrated using ions specific to each compound and used as detected amounts.

TABLE 4

| | |
|---|---|
| Thermal Desorption Unit | TDU (Gerstel Inc.) |
| Thermal Desorption Temperature | 25° C. (0.5 min) → 720° C./min → 230° C. (5 min) |
| Cryofocus | −10° C. (0.5 min) → 720° C./min → 240° C. (10 min) |
| GCMS Device | 6890GC/5975MS (Agilent) |
| Column | DB-WAX UI, inner diameter 0.25 mm × film thickness |
| | 0.25 μm × length 30 m |
| Oven | 40° C. (2.5 min) → 5° C./min → 80° C. → 10° C./min → |
| | 120° C. → 20° C./min → 240° C. (5 min) |
| Helium Gas | 1 mL/min |
| Scan range | m/z 33 to 300 |

### Headspace Solid-Phase Microextraction Method:

The above vial was heated at 60° C. and then retained for 40 minutes to allow the aroma compounds in the headspace to be adsorbed on the solid phase (SUPELCO SPME, 50/30 μm DVB/CAR/PDMS). For GC/MS analysis, Agilent GC 7690B and MS 5977A (Agilent Technologies, Inc.) were used, and the column used was DB-WAX UI (0.25 mm ×0.25 μm×30 M) (Agilent Technologies, Inc.). The analysis conditions for GC/MS were such that after retaining at 40° C. for 5 minutes, the temperature was raised to 250° C. by 15° C. per minute and retained for 10 minutes.

### Sensory Evaluation

In the above fermentation time measurement, the fermented milk obtained using the bacterial strains of the combinations described in Table 1 was subjected to sensory evaluation. Specifically, the fermented milk was ice-cooled immediately after fermentation, and sensory evaluation was performed by a total of 6 trained panelists. The evaluation items were the following 10 items: sourness, sweetness, bitterness, umami, harshness/odd taste, milkiness, yogurt feeling (top (first felt) acetaldehyde scent), cheesiness (cream cheese-like), fattiness (such as butter-like), and refreshing yogurt aftertaste, each of which was evaluated according to a 7-point scale of 1 to 7. The evaluation results of 6 panelists were averaged to obtain a sensory evaluation value.

### Analysis of Organic Acids

### Formic Acid/Acetic Acid

In the above fermentation time measurement, the amounts of organic acids (formic acid and acetic acid) in the fermented milk obtained using the bacterial strains of the combinations shown in Table 1 were measured by HPLC (high performance liquid chromatography) according to the following method.

### Measuring Method

The fermented milk was diluted twice with ultrapure water and deproteinized using Carrez's reagent. After deproteinization, the supernatant was filtered through a filter vial (PVDF, 0.2 μm, 1030-19022, manufactured by THOMSON), and the amounts of formic acid and acetic acid (mM) in each fermented milk were measured under the following conditions:
Guard column: ICSep ICE ORH-801 4.0 mm i.d.×20 mm (manufactured by Tokyo Chemical Industry Co., Ltd.)
Column: Column for organic acid analysis ICSep ICE ORH-801 6.5 mm i.d.×300 mm, manufactured by Transgenomic (manufactured by Tokyo Chemical Industry Co., Ltd.), two connected
Oven temperature 55° C.
Flow rate: 0.5 mL/min
Detector: Electric conductivity detector CDD-10AVvp (manufactured by Shimadzu Corporation)
Injection volume: 10 μL
Mobile phase: 7.5 mM p-toluenesulfonic acid
Reaction solution: 7.5 mM p-toluenesulfonic acid+150 μM EDTA·2NA+30 mM Bis Tris.

### D/L-Lactic Acid

In the above fermentation time measurement, the amounts of D-lactic acid and L-lactic acid in the fermented milk obtained using the bacterial strains of the combinations shown in Table 1 were measured by HPLC (high performance liquid chromatography) according to the following method.

### Measuring Method

The fermented milk was diluted twice with ultrapure water and deproteinized using Carrez's reagent. After deproteinization, the supernatant was filtered through a filter vial (PVDF, 0.2 μm, 1030-19022, manufactured by THOMSON), and the amounts of D-lactic acid and L-lactic acid (mM) in each fermented milk were measured under the following conditions:
Guard column: SUMICHIRAL OA-5000 5 μm 4 mm i.d.×10 mm (manufactured by Sumika Chemical Analysis Service, Ltd.)
Column: SUMICHIRAL OA-5000 4.6 mm i.d.×150 mm (manufactured by Sumika Chemical Analysis Service, Ltd.)
Oven temperature: 40° C.
Flow rate: 1.0 mL/min
Detector: SPD-M20A (manufactured by Shimadzu Corporation)
Injection volume: 10 μL
Mobile phase: 2 mM CuSO_{4·5}H₂O+5% isopropanol.

### Results

### (1) All Water-Soluble Components and Sensory Evaluation

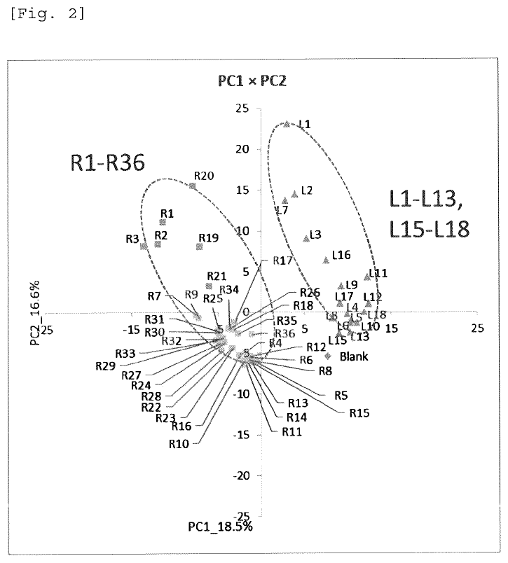

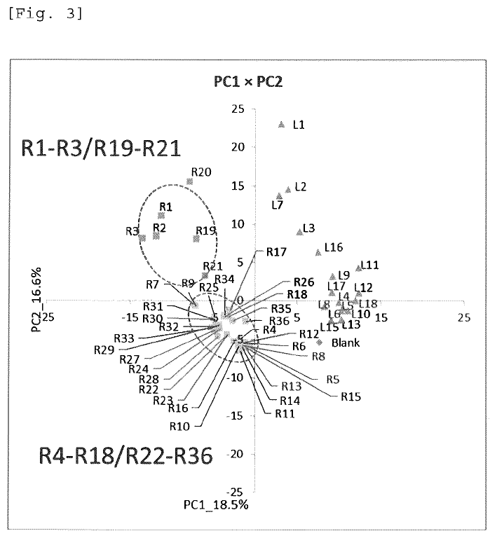

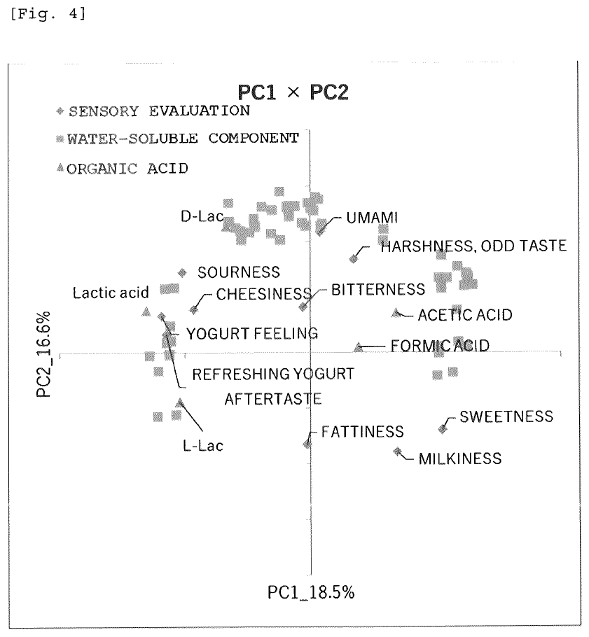

Principal component analysis (PCA) was performed on water-soluble components and sensory evaluation values for the fermented milk obtained by using S. thermophilus and lactic acid bacteria of the Lactobacillaceae in the combinations shown in Table 1. The water-soluble components (all water-soluble components) here are a combination of the water-soluble components obtained by the above water-soluble component analysis and the formic acid, acetic acid, D-Lactic acid, and L-lactic acid obtained by the above organic acid analysis. FIGS. 2 and 3 show the relationship between the first principal component score (PC1) and the second principal component score (PC2) for each fermented milk. FIG. 2 is a scatter diagram encircling the case of using S. thermophilus (R1 to R36) and the case of fermentation using lactic acid bacteria of the Lactobacillaceae alone without using S. thermophilus (L1 to L13, L15 to L18), and FIG. 3 is a scatter diagram encircling the case of using L. delbrueckii (R1 to R3/R19 to P21) and the case of using the others (R4 to R18/R22 to R36) for lactic acid bacteria of the Lactobacillaceae. Furthermore, FIG. 4 is a scatter diagram for the all water-soluble components and sensory evaluation items corresponding to plot positions of the samples of FIGS. 2 and 3.

As shown in FIG. 2, the results were divided into two in the case of using S. thermophilus (R1 to R36) and in the case of fermentation using lactic acid bacteria of the Lactobacillaceae alone without using S. thermophilus (L1 to L13, L15 to L18), suggesting that the all water-soluble components in the fermented milk and sensory evaluation values were greatly affected by the presence or absence of combination with S. thermophilus. In addition, as shown in FIG. 3, among the lactic acid bacteria of the Lactobacillaceae, the results were also divided into two in the case of using L. delbrueckii (R1 to R3/R19 to R21) and in the case of using the others (R4 to R18/R22 to R36), suggesting that the all water-soluble components in the fermented milk and sensory evaluation values were further affected by the type of lactic acid bacteria of the Lactobacillaceae, that is, L. delbrueckii or the others.

Furthermore, in FIG. 4, the results were such that the further to the left of the x-axis, the stronger the yogurt feeling (top (first felt) acetaldehyde scent) and the refreshing yogurt aftertaste (yogurt likeness), and the further to the upper left, the higher the amount of D-lactic acid (D-Lac) and the stronger the sourness. In addition, the results were such that the further to the upper right, the more acetic acid resulting in strong harshness and odd taste, and the further to the lower right, the stronger the milkiness and sweetness. As shown in FIGS. 2 and 4, in the case of fermentation using lactic acid bacteria of the Lactobacillaceae alone without using S. thermophilus (L1 to L13, L15 to L18), many of them were plotted on the right side of the x-axis, especially on the upper right, and most of them were evaluated as being equivalent to unfermented milk (blank) and having almost no fermented flavor, or having a strong harshness and odd taste. Meanwhile, in the case of using S. thermophilus and lactic acid bacteria of the Lactobacillaceae in combination (R1 to R36), they were plotted on the left side of the x-axis from they lower left to the upper left, and were evaluated as having a strong yogurt feeling (top acetaldehyde scent) and a refreshing yogurt aftertaste. In addition, from FIGS. 3 and 4, the fermented milks fermented with S. thermophilus and L. delbrueckii (R1 to R3 /R19 to R21) were plotted in the upper left with strong sourness. Meanwhile, the fermented milks fermented with S. thermophilus and lactic acid bacteria of the Lactobacillaceae other than L. delbrueckii (R4 to R18/R22 to R35) were plotted on the lower left side of the origin, and were evaluated to have weaker sourness as well as stronger milkiness and sweetness than the fermented milks fermented using S. thermophilus and L. delbrueckii.

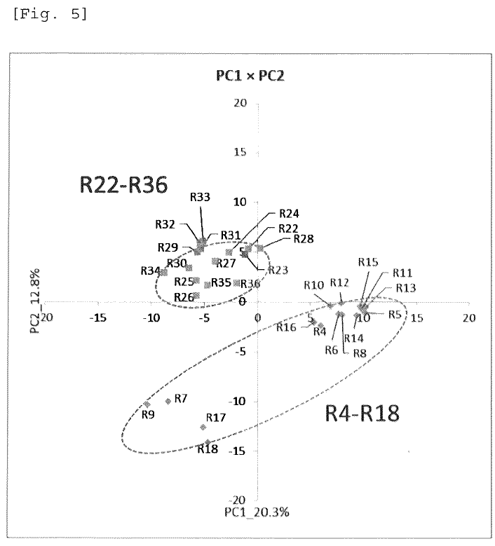

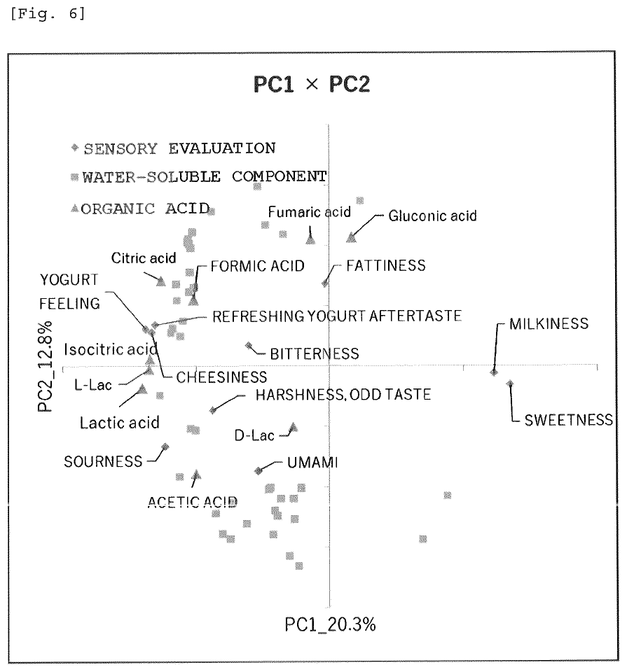

Furthermore, principal component analysis (PCA) was also performed on the all water-soluble components and sensory evaluation values in the case of using lactic acid bacteria of the Lactobacillaceae other than L. delbrueckii (R4 to R18, R22 to R36). FIG. 5 shows a scatter diagram showing the relationship between the first principal component score (PC1) and the second principal component score (PC2) for each fermented milk. FIG. 5 is a scatter diagram encircling the case of using S. thermophilus prtS(−) (R4 to R18) and the case of using S. thermophilus prtS(+) (R22 to R36). Furthermore, FIG. 6 shows a scatter diagram for the all water-soluble components and sensory evaluation items corresponding to plot positions of the samples in FIG. 5.

As shown in FIG. 5, among S. thermophilus, the results were divided into two in the case of carrying the prtS gene (prtS(+); R22 to R36) and in the case of not carrying the prtS gene (prtS(−): R4 to R18), suggesting that the all water-soluble components in the fermented milk and sensory evaluation values were further affected also by the presence or absence of carrying the prtS gene of S. thermophilus.

Further, in FIG. 6, the further to the right, the stronger the milkiness and sweetness, and the further to the left, the more L-lactic acid (L-Lac) and the stronger yogurt likeness, and the further to the lower left, the more acetic acid and D-lactic acid (D-Lac) and the stronger sourness and harshness. From FIGS. 5 and 6, in the case of using S. thermophilus not carrying the prtS gene (prtS(−)), most of the samples were located in the lower right and some of the samples were plotted in the lower left direction. On the other hand, in the case of using S. thermophilus carrying the prtS gene (prtS(+)), they were plotted in the upper left direction, suggesting that prtS(+) was less in sourness and harshness than prtS(−) and had a yogurt likeness flavor with a strong top acetaldehyde scent.

### (2) Aroma Compounds and Sensory Evaluation

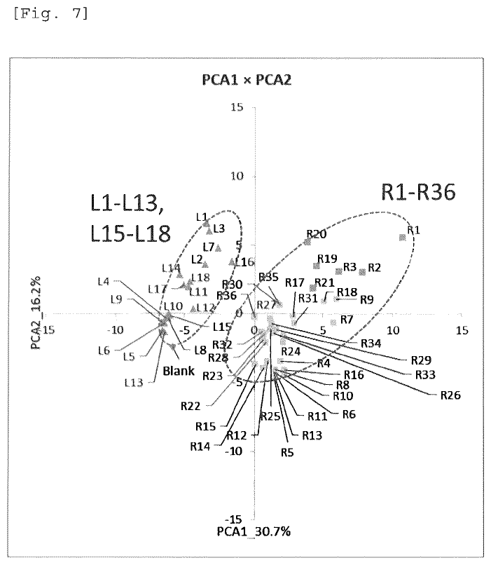

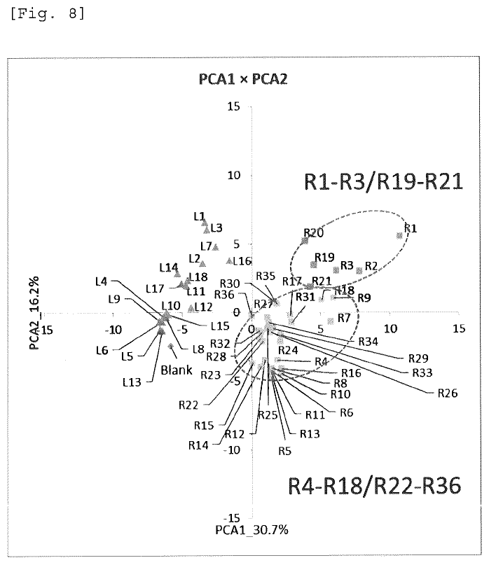

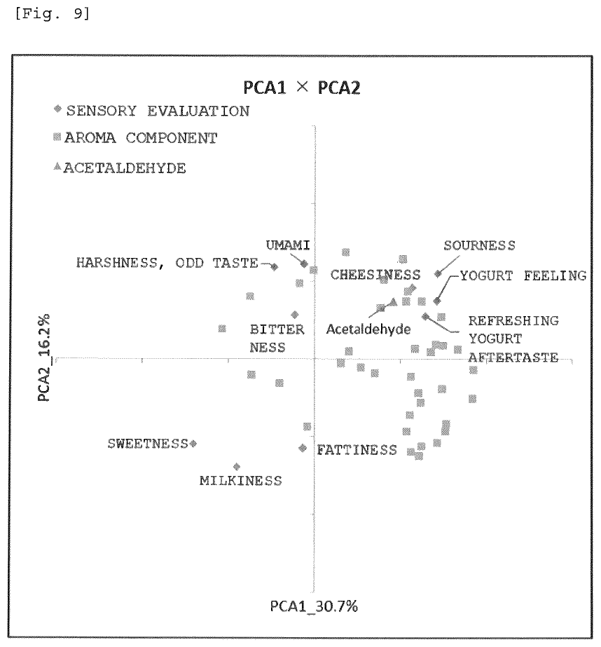

Principal component analysis (PCA) was performed on aroma compounds and sensory evaluation values for the fermented milk obtained by using S. thermophilus and lactic acid bacteria of the Lactobacillaceae in the combinations shown in Table 1. FIGS. 7 and 8 show the relationship between the first principal component score (PC1) and the second principal component score (PC2) for each fermented milk. FIG. 7 is a scatter diagram encircling the case of using S. thermophilus (R1 to R36) and the case of fermentation using lactic acid bacteria of the Lactobacillaceae alone without using S. thermophilus (L1 to L13, L15 to L18), and FIG. 8 is a scatter diagram encircling the case of using L. delbrueckii (R1 to R3/R19 to R21) and the case of using the others (R4 to R18/R22 to R36) for lactic acid bacteria of the Lactobacillaceae. Furthermore, FIG. 9 is a scatter diagram for the aroma compounds and sensory evaluation items corresponding to plot positions of the samples of FIGS. 7 and 8.

As shown in FIG. 7, the results were divided into two in the case of using S. thermophilus (R1 to R36) and in the case of fermentation using lactic acid bacteria of the Lactobacillaceae alone without using S. thermophilus (L1 to L13, L15 to L18), suggesting that the aroma compounds in the fermented milk and sensory evaluation values were greatly affected by the presence or absence of combination with S. thermophilus. In addition, as shown in FIG. 8, among the lactic acid bacteria of the Lactobacillaceae, the results were also divided into two in the case of using L. delbrueckii (R1 to R3/R19 to R21) and in the case of using the others (R4 to R18/R22 to R36), suggesting that the aroma compounds in the fermented milk and sensory evaluation values were further affected by the type of lactic acid bacteria of the Lactobacillaceae, that is, L. delbrueckii or the others.

In FIG. 9, the results were such that the further to the upper right, the stronger the sourness, acetaldehyde and yogurt feelings (top acetaldehyde scent), and refreshing yogurt aftertaste (yogurt likeness), and the further to upper left, the stronger the harshness and odd taste, and the further to the lower left, the stronger the milkiness and sweetness. As shown in FIGS. 7 and 9, in the case of fermentation using lactic acid bacteria of the Lactobacillaceae alone without using S. thermophilus (L1 to L13, L15 to L18), they were plotted from the lower left to the upper left, and were evaluated as being equivalent to unfermented milk (blank) and having almost no fermented flavor, or having a strong harshness and odd taste. Meanwhile, in the case of using S. thermophilus and lactic acid bacteria of the Lactobacillaceae in combination (R1 to R36), they were plotted from the lower right to the upper right, and were evaluated as having a weak harshness and odd taste, a strong yogurt feeling (top acetaldehyde scent) and a refreshing yogurt aftertaste, and yogurt likeness. In addition, from FIGS. 8 and 9, the fermented milks fermented with S. thermophilus and L. delbrueckii (R1 to R3/R19 to R21) were plotted to the upper right of the origin. Meanwhile, the fermented milks fermented with S. thermophilus and lactic acid bacteria of the Lactobacillaceae other than L. delbrueckii (R4 to R18/R22 to R36) were plotted to the lower right of the origin and to the lower left of the fermented milks fermented with S. thermophilus and L. delbrueckii. That is, the fermented milks fermented with S. thermophilus and lactic acid bacteria of the Lactobacillaceae other than L. delbrueckii were evaluated as having weaker sourness as well as stronger milkiness and sweetness than the fermented milks fermented with S. thermophilus and L. delbrueckii.

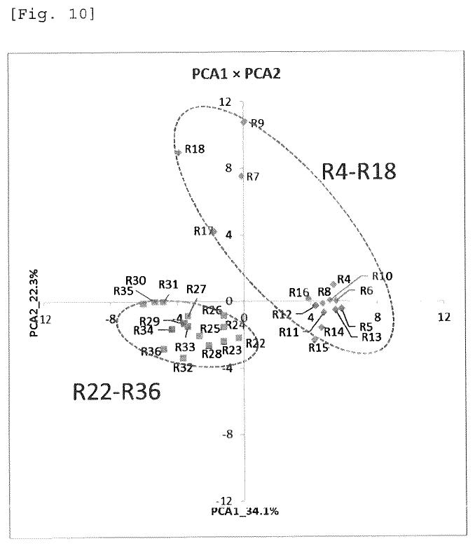

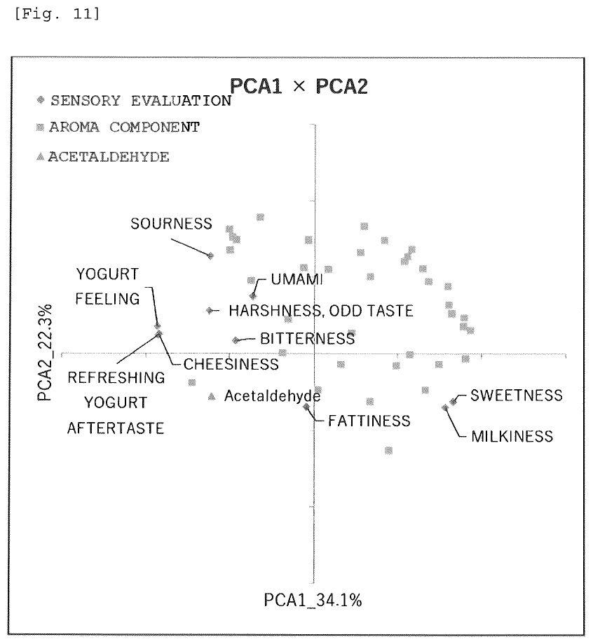

Furthermore, principal component analysis (PCA) was also performed on the aroma compounds and sensory evaluation values in the case of using lactic acid bacteria of the Lactobacillaceae other than L. delbrueckii (R4 to R18, R22 to R36). FIG. 10 shows a scatter diagram showing the relationship between the first principal component score (PC1) and the second principal component score (PC2) for each fermented milk. FIG. 10 is a scatter diagram encircling the case of using S. thermophilus prtS(−) (R4 to R18) and the case of using S. thermophilus prtS(+) (R22 to R36). Furthermore, FIG. 11 shows a scatter diagram for the aroma compounds and sensory evaluation items corresponding to plot positions of the samples in FIG. 10.

As shown in FIG. 10, among S. thermophilus, the results were divided into two in the case of carrying the prtS gene (prtS(+): R22 to R36) and in the case of not carrying the prtS gene (prtS(−): R4 to R18), suggesting that the aroma compounds in the fermented milk and sensory evaluation values were further affected also by the presence or absence of carrying the prtS gene of S. thermophilus.

Further, in FIG. 11, the further to the left of the x-axis, the stronger the yogurt likeness, and the further to the lower left, the more acetaldehyde, and the further to the upper left, the stronger the sourness, harshness, and odd taste, the further to the lower right, the stronger the sweetness and milkiness. From FIGS. 10 and 11, in the case of using S. thermophilus not carrying the prtS gene (prtS(−)), some of the samples were plotted in the upper left direction, but most of the samples were located on the right side of the x-axis, and in the case of using S. thermophilus carrying the prtS gene (prtS(+)), they were plotted on the left side of the x-axis, especially on the lower left. This suggested that prtS(+) had a flavor stronger in yogurt likeness than prtS(−), but weaker in sourness, harshness, and odd taste.

### Test Example 2

### S. thermophilus

As S. thermophilus, the same bacterial strains as the bacterial strains used in Test Example 1 (S. thermophilus prtS(−): “S. thermophilus 1131”; S. thermophilus prtS(+): “S. thermophilus NITE BP-02875”) shown in Table 5 below were used.

### Lactic Acid Bacteria of the Lactobacillaceae

As lactic acid bacteria of the Lactobacillaceae, the bacterial strains of the bacterial species shown in Table 5 below were used. In Table 5, strains whose bacterial strain numbers are indicated by JCM were obtained from RIKEN BioResource Research Center Microbe Division (http://jcm.brc.riken.jp/ja/), strains whose strain numbers are indicated by NBRC were obtained from National Institute of Technology and Evaluation Biological Resource Center (http://www.nite.go.jp/nbrc/), and strains whose strain numbers are indicated by NCIMB were obtained from the United Kingdom National Culture Collection, the National Collection of Industrial, Food and Marine Bacteria.

### Fermentation Time Measurement

The above S. thermophilus and lactic acid bacteria of the Lactobacillaceae were used in the combinations shown in Table 5 below (Examples: R100 to R159, Comparative Examples: L100 to L129) to measure the time required for fermentation.

TABLE 5

| | | S. thermophilus | | |
|---|---|---|---|---|
| | | | prts ( +) | |
| Lactic Acid Bacteria of the Lactobacillaseae | | prts (−) | NITE | |
| Bacterial Species Name | Bacterial Species Number | 1131 | BP-02875 | None |
| Lactobacillus acidophilus | JCM 1132^{T} | R100 | R130 | L100 |
| Lactobacillus amylovorus | JCM 1126^{T} | R101 | R131 | L101 |
| Lactobacillus crispatus | JCM 1185^{T} | R102 | R132 | L102 |
| Lactobacillus gasseri | JCM 1131^{T} | R103 | R133 | L103 |
| Lactobacillus helveticus | JCM 1120^{T} | R104 | R134 | L104 |
| Lactobacillus johnsonii | JCM 2012^{T} | R105 | R135 | L105 |
| Lactobacillus kefiranofaciens | JCM 6985^{T} | R106 | R136 | L106 |
| subsp. kefiranofaciens | | | | |
| Lactobacillus paragasseri | JCM 5343^{T} | R107 | R137 | L107 |
| Lacticaseibacillus casei | JCM 1134^{T} | R108 | R138 | L108 |
| Lacticaseibacillus paracasei | NBRC 15889^{T} | R109 | R139 | L109 |
| subsp. paracasei | | | | |
| Lacticaseibacillus rhamnosus | JCM 1136^{T} | R110 | R140 | L110 |
| Latilactobacillus sakei | JCM 1157^{T} | R111 | R141 | L111 |
| Liquorilactobacillus | JCM 12392^{T} | R112 | R142 | L112 |
| satsumensis | | | | |
| Liquorilactobacillus cacaonum | P2001810 | R113 | R143 | L113 |
| Ligilactobacillus salivarius | JCM 1231^{T} | R114 | R144 | L114 |
| Lactiplantibacillus plantarum | NCIMB 11974^{T} | R115 | R145 | L115 |
| Lactiplantibacillus | NCIMB 13579^{T} | R116 | R146 | L116 |
| paraplantarum | | | | |
| Lactiplantibacillus pentosus | NCIMB 8026^{T} | R117 | R147 | L117 |
| Limosilactobacillus fermentum | JCM 1173^{T} | R118 | R148 | L118 |
| Limosilactobacillus reuteri | JCM 1112^{T} | R119 | R149 | L119 |
| Levilactobacillus brevis | JCM 1059^{T} | R120 | R150 | L120 |
| Levilactobacillus namurensis | NBRC 107158^{T} | R121 | R151 | L121 |
| Lentilactobacillus buchneri | NCIMB 8007^{T} | R122 | R152 | L122 |
| Lentilactobacillus kefiri | JCM 5818^{T} | R123 | R153 | L123 |
| Lentilactobacillus | JCM 12493^{T} | R124 | R154 | L124 |
| parabuchneri | | | | |
| Pediococcus pentosaceus | JCM 5890^{T} | R125 | R155 | L125 |
| Pediococcus acidilactici | JCM 8797^{T} | R126 | R156 | L126 |
| Leuconostoc lactis | JCM 6123^{T} | R127 | R157 | L127 |
| Leuconostoc mesenteroides | JCM 6124^{T} | R128 | R158 | L128 |
| subsp. mesenteroides | | | | |
| Leuconostoc | JCM 9696^{T} | R129 | R159 | L129 |
| pseudomesenteroides | | | | |

| | |
|---|---|
| None | Blank |

For fermentation, 0.5% (0.5 mL/100 mL, hereinafter the same) of each bacterial strain or a combination thereof (S. thermophilus: lactic acid bacteria of the Lactobacillaceae=about 1:1 (bacteria count)) was added to a medium obtained by adding 0.1% yeast extract to a 10% skim milk medium so that the amount of each bacterial strain was 0.5% (1% in total), which was fermented at 37° C. for 24 hours (aerobic, stationary culture) to obtain each fermented milk. The time from the addition of each bacterial strain until the pH became 4.5 or less was measured and used as the fermentation time required for fermentation. If the pH did not reach 4.5 within 24 hours after the addition. of bacterial strain, the fermentation was terminated with the fermentation time set to 724 hours (1440 minutes).

As a result of fermentation time measurement, it was confirmed that when S. thermophilus was used (R100 to R159), the p-value was less than 0.01 in each case and thus the fermentation time was significantly shortened compared to when fermentation was performed using lactic acid bacteria of the Lactobacillaceae alone without using S. thermophilus (L100 to L129), regardless of carrying the prtS gene. Table 6 below shows fermentation time (min) when using S. thermophilus carrying the prtS gene (R130 to R159) and when fermenting with lactic acid bacteria of the Lactobacillaceae alone (L100 to 1129).

TABLE 6

| | Fermentation | | Fermentation |
|---|---|---|---|
| Combination | Time | Combination | Time |
| No . | [min] | No. | [min] |
| R130 | 610 | L100 | 1015 |
| R131 | 520 | L101 | 1440 |
| R132 | 580 | L102 | 1440 |
| R133 | 525 | L103 | 1440 |
| R134 | 435 | L104 | 675 |
| R135 | 430 | L105 | 1440 |
| R136 | 510 | L106 | 1440 |
| R137 | 535 | L107 | 1440 |
| R138 | 460 | L108 | 1440 |
| R139 | 500 | L109 | 1440 |
| R140 | 580 | L110 | 1130 |
| R141 | 495 | L111 | 1440 |
| R142 | 535 | L112 | 1055 |
| R143 | 525 | L113 | 1440 |
| R144 | 555 | L114 | 1440 |
| R145 | 495 | L115 | 1440 |
| R146 | 440 | L116 | 1440 |
| R147 | 435 | L117 | 1440 |
| R148 | 535 | L118 | 1440 |
| R149 | 485 | L119 | 1440 |
| R150 | 450 | L120 | 1440 |
| R151 | 530 | L121 | 1440 |
| R152 | 615 | L122 | 1440 |
| R153 | 495 | L123 | 1440 |
| R154 | 595 | L124 | 1440 |
| R155 | 420 | L125 | 1440 |
| R156 | 565 | L126 | 1440 |
| R157 | 555 | L127 | 1235 |
| R158 | 455 | L128 | 1440 |
| R159 | 525 | L129 | 1440 |
| Average | 513.0 | Average | 1370.3 |
| SD | 55.4 | SD | 176.8 |

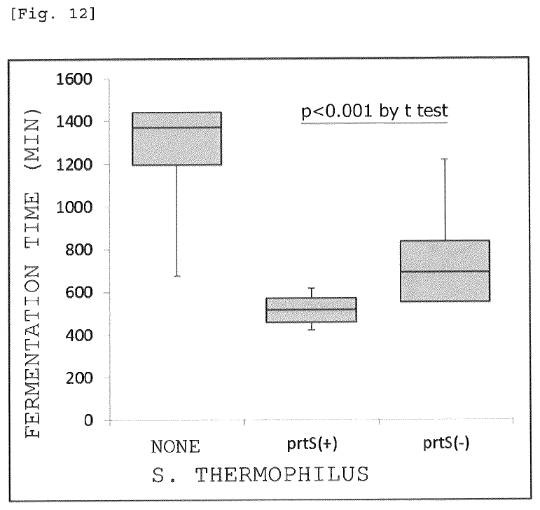

In addition, FIG. 12 shows fermentation time (mean values, min) when using S. thermophilus not carrying the puts gene (prtS(−), R100 to R129 (n=30)), when using S. thermophilus carrying the prtS gene (prtS(+), R130 to R159 (n=30)), and when not using S. thermophilus (none, L100 to L129 (n=30)). As a result of the t-test, the results for prtS(+) had p-values of less than 0.001 with respect to the results for prtS(−). From the above, as in Test Example 1, it was confirmed that prtS(+) shortened the fermentation time more significantly than prtS(−).

### INDUSTRIAL APPLICABILITY

As described above, the present invention makes it possible to provide a novel lactic acid bacteria starter that can stably produce fermented milk that completes fermentation in a sufficiently short time and has a flavor characteristic differentiated from conventional fermented milk, “mild flavor with low sourness but with milkiness and sweetness”, a method for producing fermented milk using the same, and fermented milk obtained thereby,

### Accession Number

1. (1) identification label: Streptococcus thermophilus OLS4496
(2) accession number: NITE BP-02875
(3) date of accession: Feb. 5, 2019
(4) depositary institution: National Institute of Technology and Evaluation Patent Microorganisms Depositary 2.
(1) Identification label: Lactobacillus paracasei subsp. paracasei OLL204220
(2) Accession number: NITE BP-02244
(3) Date of accession: Apr. 25, 2016
(4) Depositary institution: National Institute of Technology and Evaluation Patent Microorganisms Depositary
(1) Identification label: Lactobacillus delbrueckii subspecies bulgaricus OLL11255
(2) Accession number: NITE BP-76
(3) Date of original deposit: Feb. 10, 2005 (date of transfer to deposit under the Budapest Treaty: Apr. 1, 2009)
(4) Depositary institution: National Institute of Technology and Evaluation Patent Microorganisms Depositary 4.
(1) Identification label: Lactobacillus delbrueckii OLL204989
(2) Accession number: NITE BP-02874
(3) Date of accession: Feb. 5, 2019
(4) Depositary institution: National Institute of Technology and Evaluation Patent Microorganisms Depositary

### Sequence Listing Free Text

SEQ ID NO: 1
<223> forward primer
SEQ ID NO 2
<223> reverse primer

1. A lactic acid bacteria starter comprising: Streptococcus thermophilus; and a lactic acid bacterium of the Lactobacillaceae other than Lactobacillus delbrueckii.

2. The lactic acid bacteria starter according to claim 1, wherein the Streptococcus thermophilus is a bacterium carrying a prtS gene.

3. The lactic acid bacteria starter according to claim 1, wherein the lactic acid bacterium of the Lactobacillaceae is at least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

4. The lactic acid bacteria starter according to claim 1, wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.

5. A method for producing fermented milk, comprising: a fermentation step of adding the lactic acid bacteria starter according to claim 1 to a milk preparation solution containing raw material milk and fermenting the mixture to obtain fermented milk.

6. A method for producing fermented milk, comprising: a fermentation step of adding Streptococcus thermophilus and a lactic acid bacterium of the Lactobacillaceae other than Lactobacillus delbrueckii to a milk preparation solution containing raw material milk and fermenting the mixture to obtain fermented milk.

7. The method for producing fermented milk according to claim 6, wherein the Streptococcus thermophilus is a strain carrying a prtS gene.

8. The method for producing fermented milk according to claim 6, wherein the lactic acid bacterium of the Lactobacillaceae is at least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

9. The method for producing fermented milk according to claim 6, wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.

10. A fermented milk comprising: Streptococcus thermophilus; and a lactic acid bacterium of the Lactobacillaceae other than Lactobacillus delbrueckii.

11. The fermented milk according to claim 10, wherein the Streptococcus thermophilus is a strain carrying a prtS gene.

12. The fermented milk according to claim 10, wherein the lactic acid bacterium of the Lactobacillaceae is at least one selected from the group consisting of lactic acid bacteria in the genus Lactobacillus, lactic acid bacteria in the genus Lacticaseibacillus, lactic acid bacteria in the genus Lactiplantibacillus, lactic acid bacteria in the genus Liquorilactobacillus, lactic acid bacteria in the genus Latilactobacillus, lactic acid bacteria in the genus Ligilactobacillus, lactic acid bacteria in the genus Limosilactobacillus, lactic acid bacteria in the genus Lentilactobacillus, lactic acid bacteria in the genus Levilactobacillus, lactic acid bacteria in the genus Pediococcus, and lactic acid bacteria in the genus Leuconostoc.

13. The fermented milk according to claim 10, wherein the lactic acid bacterium of the Lactobacillaceae is at least one selected from the group consisting of Lactobacillus gasseri, Lactobacillus paragasseri, Lactobacillus helveticus, Lactobacillus johnsonii, Lactobacillus acidophilus, Lactobacillus crispatus, Lactobacillus amylovorus, Lactobacillus kefiranofaciens, Lacticaseibacillus paracasei, Lacticaseibacillus rhamnosus, Lacticaseibacillus casei, Lactiplantibacillus plantarum, Lactiplantibacillus paraplantarum, Lactiplantibacillus pentosus, Liquorilactobacillus cacaonum, Liquorilactobacillus satsumensis, Latilactobacillus sakei, Ligilactobacillus salivarius, Limosilactobacillus fermentum, Limosilactobacillus reuteri, Lentilactobacillus buchneri, Lentilactobacillus parabuchneri, Lentilactobacillus kefiri, Levilactobacillus brevis, Levilactobacillus namurensis, Pediococcus pentosaceus, Pediococcus acidilactici, Leuconostoc lactis, Leuconostoc mesenteroides, and Leuconostoc pseudomesenteroides.


SEQUENCE LISTING (2 sequences)

SEQ ID NO: 1 - DNA, 20 bp, Artificial Sequence
  Forward primer
gacttgaagaaacagcgcgt

SEQ ID NO: 2 - DNA, 20 bp, Artificial Sequence
  Reverse primer
taggtggaggcgttacagtg